(19) United States
(12) Patent Application Publication
(10) Pub. No.: US 2016/0008636 A1
(43) Pub. Date: Jan. 14, 2016

(54) ULTRASOUND IMAGING SHEATH AND ASSOCIATED METHOD FOR GUIDED PERCUTANEOUS TRANS-CATHETER THERAPY

(71) Applicant: GUIDED INTERVENTIONS, INC., Elyria, OH (US)

(72) Inventor: Reinhard J. Warnking, Setauket, NY (US)

(21) Appl. No.: 14/770,941

(22) PCT Filed: Feb. 27, 2014
(86) PCT No.: PCT/US2014/018934

§ 371 Date: Aug. 27, 2015

Related U.S. Application Data
(60) Provisional application No. 61/770,810, filed on Feb. 28, 2013.
(60) Provisional application No. 61/770,818, filed on Feb. 28, 2013.

(51) Int. Cl. A61N 7/02; A61B 5/055; A61B 8/00; A61B 8/08; A61B 8/12

(52) U.S. Cl. CPC A61N 7/022; A61B 8/0841; A61B 8/12; A61B 8/445; A61B 8/4488; A61B 8/461; A61B 8/483; A61B 5/055; A61B 2018/00351

(57) ABSTRACT

Intra-organ ultrasound images are obtained by integrating ultrasound array configurations at the distal region of a sheath or guiding catheter integral to any catheter based intervention. A dual mode ablation/imaging circular ultrasound array is used to create circular or partial circular lesions. The sites of the individual lesion segments are identified in an ultrasound 2D image. In the case of PV isolation the process of ablating individual segments identified in the ultrasound image is repeated until a circumferential, continuous lesion has been achieved and PV isolation has been confirmed with the coaxial loop sensing catheter which also serves as a guide wire.

### CROSS-REFERENCE TO RELATED APPLICATIONS

This application claims the benefit of U.S. Provisional Application No. 61/770,810 filed Feb. 28, 2013 and the benefit of U.S. Provisional Application No. 61/770,818 filed Feb. 28, 2013.

### FIELD OF THE INVENTION

The present invention relates in part to integrated ultrasound imaging with a catheter delivery sheath as used for electrophysiology (EP), interventional cardiology and interventional radiology procedures.

The present invention also relates to percutaneous catheter based treatments of various diseases as, for example, Atrial Fibrillation (AF), GERD, urinary tract disease, valve disease and lung tumors in mammalian subjects.

### BACKGROUND OF THE INVENTION

Ultrasound imaging is well established to guide interventional procedures. Ultrasound imaging has the advantage that real time guidance with morphological information (unlike with fluro guidance which does not provide morphological information) is obtained without radiation burden. However, today's ultrasound imaging catheters do not provide simultaneous guidance relative to the intervention or therapy if the imaging catheter is exchanged for the treatment catheter. For many procedures either the therapy catheter is inserted or the ultrasound imaging catheter. Therefore, the image guidance cannot be obtained simultaneously to the therapeutic action. If the anatomy allows, both, imaging as well as treatment catheter can be inserted to obtain real time or simultaneous guidance. However, this requires an additional puncture for the imaging catheter.

A typical example for the above situation is abdominal aortic aneurysm (AAA) repair. An imaging run is performed to confirm graft selection and planning of placement. Then the imaging catheter is withdrawn and the treatment catheter (in this case carrying the graft) is inserted and the graft is deployed. After the deployment an imaging run is performed to confirm correct placement (i.e. mechanical stability) and proper expansion (i.e. lack of leaks). It would be desirable to obtain the ultrasound imaging guidance simultaneously with the therapeutic procedure, i.e. without having to perform a diagnostic/therapeutic catheter exchange. This way the procedure would be optimized and much easier to perform.

Many heart disease conditions are treated by guidance with Intra Cardiac Echocardiography (ICE) catheter imaging as, for example, catheter ablation to treat Atrial Fibrillation (AF) or appendage closure. Many more treatments are evolving like percutaneous valve repair procedures which greatly benefit from ultrasound imaging guidance. Currently, percutaneous valve repair procedures utilize Trans Esophageal Echocardiography (TEE) imaging for guidance due to the lack of high quality ICE imaging and 3D ICE imaging.

The current ICE imaging is limited to 2 dimensional imaging with rather limited image quality. Two approaches utilized are phased array all electronic imaging and mechanically rotating imaging. The mechanical approach utilizes a rotating transducer at the distal catheter end which is limited in aperture (to the catheter diameter or less) and therefore needs to be advanced close to the ablation site (typically a pulmonary vein antrum in case of AF ablations) in the left atrium in order to obtain useful images. Consequently imaging and therapy are performed in an alternating fashion by advancing either the therapeutic or the imaging catheter unless a double trans-septal puncture and an additional percutaneous access are performed.

For phased array imaging, with larger long axis apertures, the catheter is positioned in the right atrium to image and guide ablations in the left atrium. While this approach is advantageous over the mechanical approach because it allows for simultaneous therapeutic action under image guidance, there is a need for better image quality in particular in the far field where the catheter ablation takes place in the case of left pulmonary vein isolations. In addition the long axis imaging format makes orientation difficult which requires a significant learning curve for electronic ICE imaging.

U.S. Pat. No. 5,135,001 proposes to obtain ultrasound image guidance through a removable circular transducer section attached to a medical instrument. This type of imaging device will not be isometric and increases the instrument diameter significantly. Also cable management from the imaging sensor(s) to the ultrasound instrument is challenging. Other proposals suggest the use of an additional lumen in the sheath to advance an imaging catheter which of course increases the overall sheath diameter significantly (see U.S. Pat. No. 5,201,315 describing a sheath with three lumens to accommodate guide wire, probe and imaging catheter).

Perhaps for these reasons, none of these proposals have been widely adopted.

With respect to the treatment of cardiac disease states such as atrial fibrillation (AF), it is noted that humans and other mammals have a four-chambered heart. Blood from the body flows into the right atrium, and from the right atrium through the tricuspid valve to the right ventricle. The right ventricle pumps the blood through the pulmonary arteries to the lungs. Blood from the lungs returns through the pulmonary veins to the left atrium, and flows from the left atrium through the mitral valve, into the left ventricle. The left ventricle, in turn, pumps the blood through the body. As the heart beats, the atria contract to pump the blood into the ventricles, and then the ventricles contract, during a phase of the heart rhythm referred to as “systole,” to pump the blood through the lungs and through the body.

For proper pumping action, the atria as well as ventricles need to contract in an organized synchronized fashion. Atrial fibrillation diminishes the pumping action of the heart.

Atrial fibrillation is a common problem with high healthcare consumption and increased morbidity and mortality.

As disclosed, for example, in U.S. Patent Application Publication No. 2009/0228003A1 or U.S. Pat. No. 7,326,201 B2, an electrode or ultrasonic transducer is advanced into the heart and actuated so as to heat the pulmonary vein annulus. It is difficult though to provide such accurate positioning of a transducer or RF electrode within a beating heart.

Numerous patents and patent applications describe the advantages of ultrasound over other energy forms, mainly radio frequency (RF). The advantage lies in the non thrombogenic nature of ultrasound which makes non contact tissue ablation possible. See US Patent Application Publication No. 2011/0137298A1; U.S. Pat. No. 7,950,397B2; US Patent Application Publication No. 2006/0064081A1; U.S. Pat. No. 7,285,116E2.

A trend can be observed to make the ablation process easier by applying a complete lesion shape instantaneously rather than forming the lesion shape through a point by point ablation procedure. See, for example, U.S. Pat. No. 7,326,201B2. Unfortunately a fixed, complete lesion shape does not completely fit all anatomic variations. Also, the risk of collateral damage is increased since these lesion shapes are rather fixed (i.e. balloon shapes) and therewith do not avoid energy deposition into collateral structures. One prominent example is phrenic nerve injury in case of RSPV ablation with balloon based systems. Anther example is esophageal injury in case of left pulmonary vein (PV) isolations. Perhaps for these reasons, none of these proposals has been widely adopted.

Many techniques have been proposed to improve catheter orientation, i.e., electromagnetic mapping techniques as commercialized by BioSense Webster or mapping combined with imaging. US Patent Application Publication No. US2008/0255449A1 assigned to ProRhythm, Inc., proposes to combine ultrasound imaging into the ablation catheter.

As far as valve repair is concerned, as disclosed, for example, in U.S. Pat. Nos. 6,306,133; 6,355,030; 6,485,489; 6,669,687; 7,229,469; and Int'l Applications PCT/US2003/008192 and PCT/US2007/087501, it has been proposed to insert a catheter-like device bearing a transducer such as an electrode or ultrasonic transducer into the heart and actuate the transducer so as to heat the, mitral annulus, denature the collagen fibers which constitute the annulus, and thereby shrink the annulus. In theory, such a procedure could bring about shrinkage of the annulus and repair mitral insufficiency. However, all of these proposals involve positioning of one or more transducers in contact with the mitral annulus during the procedure. It is difficult to provide such accurate positioning of a transducer within a beating heart. Although it is possible to momentarily halt the heartbeat, perform the procedure and then restart the heart, this adds considerable risk to the procedure. Moreover, localized heating of the annulus by a transducer in contact with the annulus introduces the further risk of damage to the epithelial cells overlying the annulus with attendant risk of thrombus formation after the procedure.

Perhaps for these reasons, none of these proposals has been widely adopted. An improvement to bringing the ultrasound transducer in indirect contact with the mitral annulus is described in U.S. Provisional Patent Application 61/204,744 by ProRhythm Inc. In this application direct contact is not required and the ultrasound transducer is positioned by means of a positioning balloon centrally in the posterior/lateral portion of the mitral annulus. However, also this approach involves potential collateral damage because of the difficulty of limiting catheter movement and therewith unwanted energy deposition superior and inferior to the mitral annulus. Besides this collateral energy deposition there is always the risk of damaging the mitral leaflets and chordae tendinae by unintentional energy deposition. Also, since the energy is directed from the inside of the heart outward there is always a potential for collateral damage in neighboring organs or structures, for example, AV node damage or atrio-esophageal fistulae. Therefore, it would be desirable to deposit heat in the mitral annulus under real time image guidance with energy selection based on target tissue distance and thickness.

### SUMMARY OF THE INVENTION

The present invention aims in part to generate high quality 2D images and 3D images in an all-electronic fashion by integrating an imaging transducer array into the distal end of a catheter delivery sheath. Pursuant to the invention, a separate imaging catheter does not need to be inserted and image guidance can be obtained simultaneously to the therapy through sheath manipulation. This aspect of the invention is cost wise advantageous and provides also from a procedure time and convenience point of view significant advantages, since a separate percutaneous access for the imaging catheter is not needed.

The present invention recognizes that the prior art catheter based ultrasound imaging technique limits the size of the imaging catheter (diameter) to the inner sheath diameter and therewith the image quality which is greatly determined by the aperture which is limited by the catheter diameter. Accordingly, the present invention contemplates the mounting of a circular ultrasound imaging array on the outside of the sheath at the distal end. Such a structure provides the largest possible aperture (given a certain access diameter) and therewith the best possible image quality and penetration.

The present invention contemplates 3D imaging which makes instrument orientation much easier and shortens the learning curve.

For intra-cardiac procedures the sheath desirably is advanced into the right atrium, for example, to guide AF ablation procedures. In case of interventional radiological procedures the sheath is advanced into the organ to be treated as for example, the aorta, for AAA repair procedures. As long as blood filled organs are examined and (or) treated, blood will provide for acoustic coupling for the ultrasound waves emitted and received by the transducer. In the case where organs not filled with blood are treated (for example, Endo Bronchial Ultrasound Procedures, EBUS) a coupling fluid is injected through the sheath (special side holes next to the transducer array might be advantageous).

The right atrial position in case of intra-cardiac procedures allows the user to obtain real time guidance of the trans-septal puncture as well as the catheter ablation itself. The image quality in particular in the far field will be advantageous compared to catheter based imaging due to the increased aperture size.

Additionally, the sheath can be advanced into the left atrium so that the imaging array is positioned inside the left atrium which will allow for different cross sectional imaging planes as well as near field imaging with improved image quality vs. far field imaging.

Yet, another aspect of the invention provides for shorter and less invasive procedures since there is no need for a separate imaging catheter which, for simultaneous imaging, does require a separate percutaneous puncture.

The apparatus of this invention most desirably includes a therapy catheter delivery sheath having proximal and distal ends, and a sheath steering structure carried on the sheath and operative to selectively bend the distal region of the sheath. The distal end of the sheath is the end which is inserted into the patient first. The opposite end is the proximal sheath end. The imaging section is desirably mounted on the distal end of the sheath so that different imaging planes can be obtained by bending or steering the distal sheath section.

Another aspect of the present invention provides methods of creating lesions inside the heart under simultaneous image guidance. The present invention recognizes the need, not for separate imaging tools or combinations of ablation tools with imaging, but a combination device, providing dual mode simultaneous ablation under image guidance with flexibility to adjust the ablation parameters depth, distance, shape, based on the image information. With such a device anatomical variations can be addressed by, for example, varying lesion shape and ablation depth. By optimizing ablation parameters based on anatomic variations a high degree of efficacy can be achieved; for example, varying wall thickness requires varying energy settings for the ablation to achieve trans-mural lesions, but to avoid collateral damage through over-ablation. Also, ultrasound imaging makes the procedure safer since collateral damage can be avoided by creating lesion shapes which spare collateral structures from being ablated.

Pursuant to the present invention, the combination imaging/ablation catheter assembly is advanced preferably into the right atrium, and after septal puncture through the septum advanced into the left atrium. The step of advancing the catheter may include advancing a delivery sheath through the septum into the left atrium of the heart and steering a distal end portion of the treatment catheter into the selected pulmonary vein opening.

The method might be performed with or without a guide wire. The guide wire might be a sensing loop shaped catheter with the loop portion at the distal end and with electrodes mounted on the loop portion. This loop catheter allows monitoring the PV isolation process real time during the ablation. Depending on the positioning of the sensing loop, the electrodes can pick up electrical cardiac voltages on the distal or proximal side of a preferentially circumferential lesion. A treatment method pursuant to the present invention mechanically stabilizes the treatment catheter so that fluoroscopy time and therewith ionizing radiation can be significantly reduced. Once the catheter is placed, the operator can actually perform the ablation procedure from the control room by placing ablation markers (via cursor and/or touch screen) on the 2D ultrasound image screen.

Methods of treating AF according to a further aspect of the invention desirably include the step of preferentially applying energy to a selected cross section of the PV antrum, which section is remote from collateral structures like the esophagus. In particular, compensation for thickness variations of the PV antrum can be achieved through output power and application time adjustments. The ablation progress and the appropriate dosing of the energy are monitored preferably through ultrasound imaging from the same circular dual mode array (or a section thereof) which generates the therapeutic beam.

Another aspect of the invention provides for a duplex emitter configuration to combine imaging with therapy. In the case of ultrasound energy the simplest configuration would be a single rotatable Tx structure allowing for A mode recording of the PV antrum thickness and distance from the transducer while using the same Tx for therapeutic ultrasound application in an interleaved timing mode. A more sophisticated combination consists of a dual mode circular array Tx to allow for true 2D ultrasound imaging and therapeutic ultrasound application quasi-simultaneously (interleaved) in the same plane.

Apparatus according to this aspect of the invention most desirably further includes a delivery sheath having proximal and distal ends, and a sheath steering structure carried on the sheath and operative to selectively bend a region of the sheath. The catheter and the emitter unit desirably are constructed and arranged so that the distal region of the catheter and the emitter unit can be advanced into the left atrium of the heart through the sheath. The catheter may also include a catheter steering mechanism carried on the catheter and operative to selectively bend a region of the catheter proximal to the emitter unit. The apparatus may also include a guide-wire, the catheter being constructed and arranged so that the catheter can be advanced over the guide-wire or the guide-wire can be advanced through the catheter.

A preferred embodiment of the invention utilizes a sensing loop shaped guide-wire. Sensing electrodes are mounted on the guide-wire loop to allow for electrical measurements distal to the ablation plane to monitor the progress of the PV isolation (entrance block) or to pace with the loop electrodes (exit block).

Further objects, features, and advantages of the present invention will be more readily apparent from the detailed described embodiments set forth below, taken in conjunction with the accompanying drawings.

### BRIEF DESCRIPTION OF THE DRAWINGS

FIG. 1 is a schematic view of a distal end portion of an elongate flexible isometric (constant outer diameter) sheath, showing the placement of a circular ultrasound imaging array at the distal section of the sheath.

FIG. 2A is a schematic view of the distal end portion of the isometric sheath of FIG. 1 inside a heart, showing the sheath as used in a typical medical procedure monitoring a trans-septal puncture.

FIG. 2B is a schematic elevational view of a video monitor or display showing an image of a cardiac septum during the ultrasound-guided procedure of FIG. 2A.

FIG. 3A is a schematic isometric view of a distal end portion of another sheath monitoring a trans-septal puncture in a heart, the sheath having a longitudinal ultrasound imaging array.

FIG. 3B is a schematic elevational view of a video monitor or display showing an image of a cardiac septum during the ultrasound-guided procedure of FIG. 3A.

FIG. 4 is a view of the imaging sheath of FIG. 1 in a related operating procedure, placed inside the left atrium of a heart and monitoring catheter-mediated ablation at the left superior pulmonary vein (LSPV).

FIG. 5 is a schematic view of a distal end portion of a modified elongate flexible medical sheath, depicting additional ultrasound imaging components mounted into a wall of the isometric sheath.

FIG. 6 is a schematic longitudinal cross-sectional view of a distal end portion of another embodiment of an elongate flexible medical sheath, in accordance with the present invention, showing an annular ultrasound imaging array divided into imaging and therapeutic sections.

FIG. 7 is a schematic perspective view of an imaging/treatment catheter in accordance with the present invention, which is introduced into a patient over a circular guide wire mapping catheter.

FIG. 8 is a schematic perspective view of the imaging/treatment catheter of FIG. 7 inserted through a sheath and positioned at the left superior pulmonary vein (LSPV) inside the left atrium with a sensing loop at the distal end advanced into the LSPV.

FIG. 9 is a flow chart depicting major steps of a PV isolation process utilizing the instrument of FIGS. 7 and 8.

FIG. 10 is partially a schematic perspective view of the imaging/treatment catheter of FIGS. 7 and 8 and partially a block diagram of a control system connected to the imaging/treatment catheter.

FIG. 11 is a block diagram of selected components of an electronic control unit and image generating components of a computer unit of an apparatus in accordance with the present invention for generating ablation zones of predetermined shape on inner surfaces of hollow internal organs of a mammalian subject.

FIG. 12 is a cross-sectional view of a portion of a right bronchial branch, showing a treatment catheter advanced through a bronchoscope into the right bronchial branch.

### DETAILED DESCRIPTION

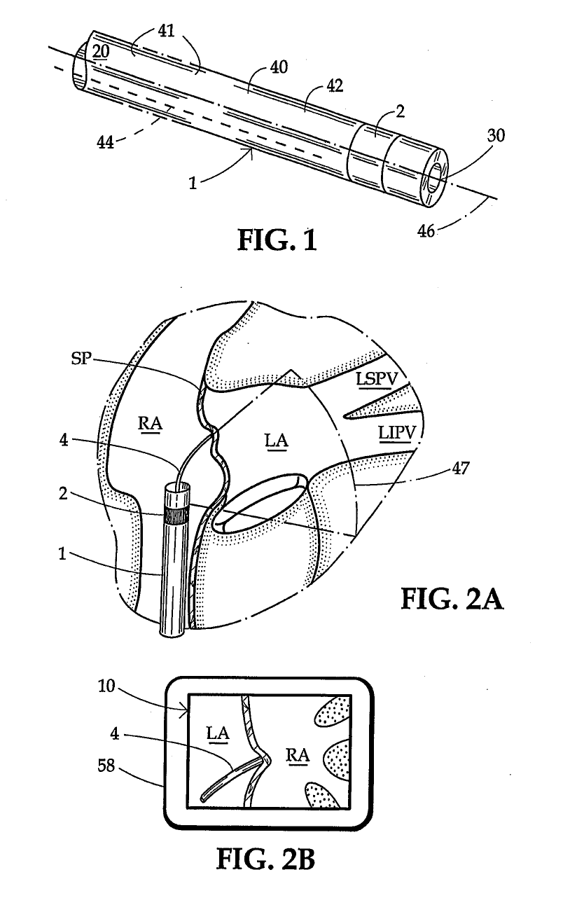

Apparatus according to one embodiment of the invention includes a sheath 1 (FIG. 1) generally in the form of an elongated tube having a proximal end 20, a distal end 30 and a proximal-to-distal axis. As used in this disclosure with reference to elongated elements for insertion into the body, the term “distal” refers to the end which is inserted into the body first, i.e., the leading end during advancement of the element into the body, whereas the term “proximal” refers to the opposite end.

Sheath 1 has an interior bore or lumen (not separately designated) extending between its proximal end 20 and its distal end 30. Desirably, sheath 1 has a relatively stiff proximal wall section 41 extending from its proximal end 20 to a juncture 40, and a relatively limber distal wall section or sheath end portion 42 extending from the juncture 40 to the distal end or tip 30. One or more pull wires 44 (only one shown) are slideably mounted in the proximal wall section 41 and connected to the distal wall section or end portion 42. The pull wire 44 is linked to a pull wire control apparatus (not shown), which can be manipulated by a physician during use of the sheath 1. By actuating the pull wire control, the physician exerts tension on the wire 44 and bends the distal end portion 42 of the sheath 1 in a predetermined or desired direction transverse to a proximal-to-distal direction or axis 46 of the sheath. The structure of sheath 1 and pull wire control may be generally as shown in U.S. Patent Application Publication No. 2006-0270976 (“the '976 Publication”), the disclosure of which is incorporated by reference herein. As discussed in greater detail in the '976 Publication, transition desirably is oblique to the proximal-to-distal axis 46 of the sheath.

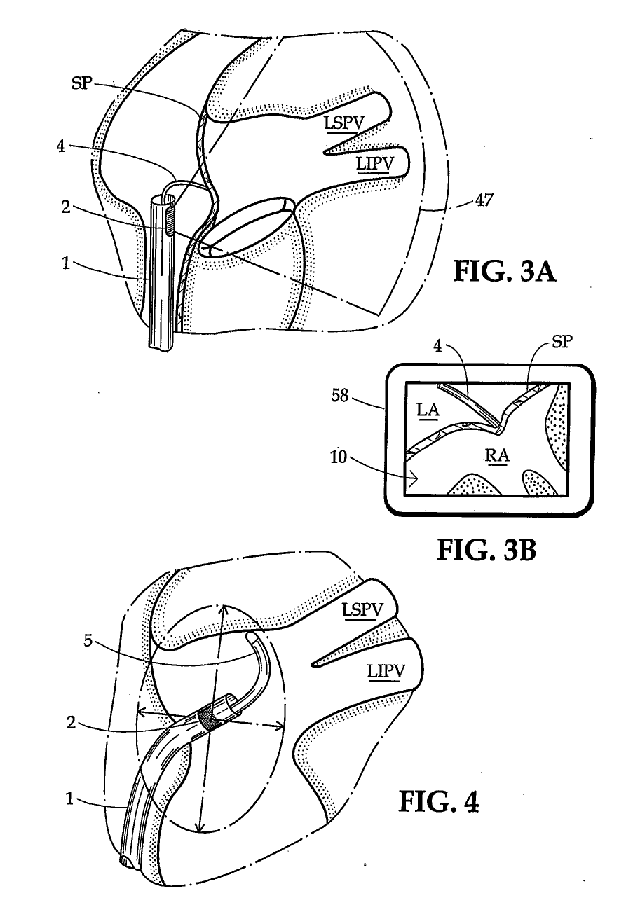

Sheath 1 desirably also is arranged so that at least the proximal section 41 is “torquable.” That is, at least the proximal section 41 of the sheath 1 is arranged to transmit torsional motion about axis 46 from the proximal end 20 (FIG. 1) along the axial extent of the sheath. Thus, by turning the proximal end 20 of the sheath 1, one can rotate the distal wall section or end portion 42 of the sheath about the proximal-to-distal axis 46. When the sheath is in a curved or bent configuration owing to tension on the pull wire 44, rotational motion of the distal wall section or end portion 42 will swing the bent section around the proximal-to-distal axis 46. Thus, by combined pulling on the pull wire 44 and rotational motion, the distal end 30 of sheath 1 and therewith an ultrasound imaging plane 47 (FIGS. 2A, 3A) can be aimed in essentially any desired direction. As disclosed in the aforementioned '976 Publication, the pull wire control can be incorporated into a handle which is physically attached to the proximal end 20 of the sheath 1. Thus, the physician can maneuver the sheath 1 by actuating the pull wire control and turning the handle, desirably with one hand, during the procedure.

The apparatus further includes, in the distal wall section or sheath end portion 42, a circular array 2 of electromechanical (e.g., PZT or piezoelectric) transducer elements for ultrasound imaging. As described above, the sheath steering allows the physician to aim the sheath distal opening (at 30) in any direction and through the same steering operation to aim the ultrasound imaging plane 47 in any direction.

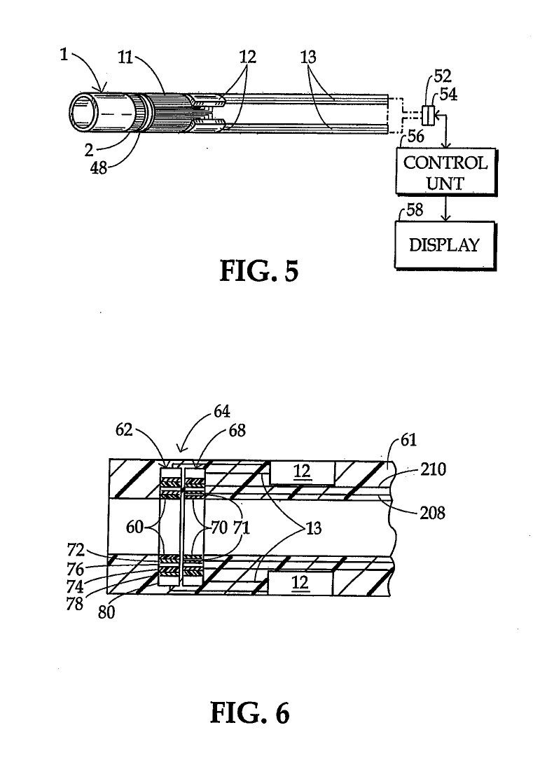

In order to keep the sheath wall reasonably thin printed flexible circuits 11 (see FIG. 5) are employed to electrically connect the ultrasound transducer array 2 with one or more multiplexer integrated circuits (ICs) 12. In one embodiment this flex circuit 11 can be an outermost sheath layer dimensioned to act as a lambda/4 impedance matching layer. The acoustic impedance of this matching layer is selected to optimize the acoustic transition from the semiconductor material of the ultrasound transducers of array to body tissue or blood: Z_{match}=SQRT(Z_{PZT}×Z_{Blood}). Preferably, several matching layers are provided. In this embodiment the ultrasound array 2, which can consist of PZT, is mounted with a die attach film 48 onto the flex circuit 11. The material of die attach film 48 (e.g., Henkel CF3350) and the thickness thereof are chosen so that the film acts as a second matching layer: Z_{MatchFilm}=SQRT(Z_{pzt}×Z_{flex}) and Z_{MatchFlex}=SQRT (Z_{film}×Z_{blood}). In an alternative embodiment the electronic circuitry is printed onto the innermost, extruded, sheath layer and then covered isometrically with an outer sheath layer which acts as one or one of several matching layers.

Another desirable feature of the present imaging sheaths is to keep the overall diameter isometric (no bulge).

In order to keep the sheath wall reasonably thin the number of connections with the ultrasound imaging console has to be minimized. Therefore a multiplexer approach is employed: with two 64:16 multiplexers 12 as shown in FIG. 5, 128 transducer elements of array 2 can be controlled with 2×16 signal lines plus supply voltage and control lines 13 running within the sheath wall from proximal end 20 to the distal end portion 42. For 3D imaging 2-dimensional arrays are required and several (n) multiplexers are employed to reduce the high array element numbers by n×64 (in case of 64:16 multiplexers).

At the proximal end the lines are terminated in a connector 52 (FIG. 5) which is mated with a connector cable 54 from a control unit 56 which feeds a video signal to an imaging console or display 58. This connector cable 52 is supplied sterile and one end placed by the sterile operator in the sterile field (to be connected to the imaging sheath) while the other end is connected to the system in the non-sterile field.

Particular attention has to be paid to the backing of array 2. For imaging purposes highly absorptive backing is desirable. This contradicts with the size requirements to keep the sheath wall acceptably thin. Accordingly, minimal backing is applied to array 2 of sheath 1. Rather than absorbing the backwards emitted ultrasound portion a diffraction layer 60 is employed to cause the backward-propagating ultrasound waves to bounce back and forth in chaotic fashion within the blood filled sheath 1. This way the backwardly emitted ultrasound is prevented from generating reverberations within the ultrasound image. Diffraction layer 60 may be made of polyimide with a conductive layer, for example, Pyralux from DuPont.

A further variation of an combined imaging/therapy sheath, depicted in FIG. 6, includes a tubular member 61 provided with a split transducer array 64, where one circular or annular section 62 is optimized for imaging with the above described diffraction mechanism (layer 60) and another circular or annular section 68 optimized for therapy. The therapy section 68 employs a metallic backing 70 to reflect a backward-propagating ultrasound wave front forward. Preferably the reflector backing 70 is spaced by a water-filled gap or distance 71 of lambda/2 behind an inner or rear surface of the transducer section 68. FIG. 6 also depicts electrodes 72, 74 sandwiching a piezoelectric or PZT layer 76, a die attach film 78, and flex circuit layer 80 in the imaging transducer section 62, with an analogous structure being present in the therapy transducer section 68. The split array configuration is described in further detail hereinafter.

Numerous other variations and combinations of the features discussed above can be utilized without departing from the present invention as defined by the claims. For example, the emitter structure can be slideably mounted within the sheath so that the sheath stays in place during the procedure. In still other arrangements, several emitters might be mounted on the sheath in a chain like fashion in order to apply energy over the length of the sheath portion inserted into the organ to be treated. Again this configuration does not require a movement of the sheath during treatment. In still other embodiments, focusing apparatus, such as lenses and diffractive elements can be employed in particular for short axis focusing of the ultrasonic energy. The right atrial position in case of intra cardiac procedures allows the user to obtain real time guidance of the trans-septal puncture as well as the catheter ablation itself.

The right atrial sheath position in case of intra cardiac procedures allows the user to obtain real time guidance of the trans-septal puncture as well as the catheter ablation itself. As depicted in FIG. 2A, sheath 1 in percutaneously inserted into the venous vascular system of a patient so that the distal wall section or sheath end portion 42 is disposed in the patient's right atrium RA. Sheath 1 carries circumferential imaging array 2. A Brockenbrough needle 4 is advanced through sheath 1 under ultrasound imaging guidance to puncture the septum SP. The user will observe the tenting effect of the needle 4 on the septum SP in the ultrasound image 10 on display 58 (FIG. 2B). This will allow the user to choose an optimal puncture site and reduce the chances for collateral damage.

FIG. 3A shows a variation of the procedure of FIG. 2A, with a sheath 72 having a longitudinal ultrasound imaging array 74. FIG. 3B shows an associated ultrasound-obtained image 10 on display 58.

All left sided cardiac interventions require a trans-septal puncture to be performed. As described above ultrasound guidance has great value since tenting of the septum clearly indicates the puncture site. Once the septum has been crossed the imaging sheath 1 can be advanced into the left atrium LA to guide the therapeutic procedure. The case of an AF treatment procedure, a distal end portion (not separately enumerated) of an ablation catheter 5 is ejected from sheath 1 and maneuvered into a pulmonary vein, e.g., left superior pulmonary vein LSPV, as shown in FIG. 4.

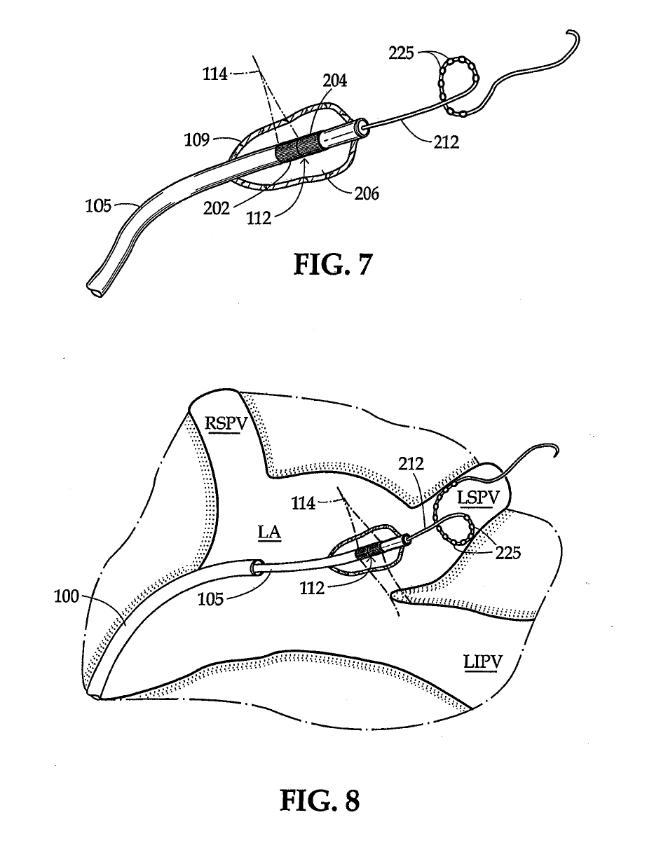

FIG. 7 illustrates related catheter-based composite imaging and therapy apparatus adapted for performing a pulmonary vein isolation procedure in treatment of atrial fibrillation. The same or similar apparatus can be used for forming annular ablations along inner surfaces of other tubular or hollow organs such as the urinary tract, the esophagus and bronchial tubes.

An expansible structure in the form of a balloon 109 (FIG. 7) is mounted to a distal end of a catheter 105. In the inflated, operative condition the balloon 109 provides a water/contrast filled volume to cool an energy emitter in case of ultrasound energy and to make it easily visible in fluoroscopy.

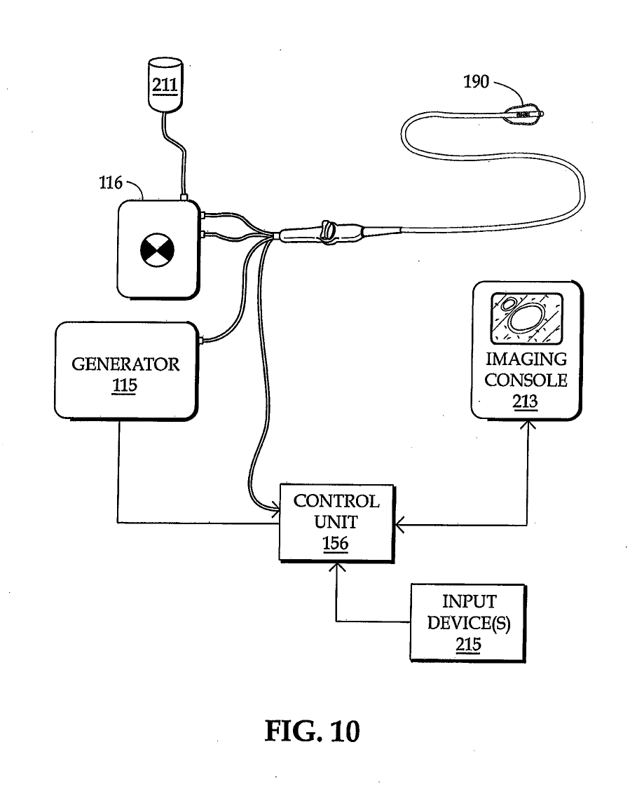

A tubular, cylindrical ultrasonic transducer array 112 is mounted to catheter 105 inside balloon 109. Transducer array 112 includes a plurality of electrically isolated and independently energizable piezoelectric or PZT transducer elements organized into a therapy transducer section 202 and an imaging transducer section 204 (FIG. 7). Therapy transducer section 202 is backed either with air or at a lamda/2 distance with a metal reflector (70, FIG. 6) in water to reflect most ultrasound energy forward or outwardly into an active beam segment 114 which will overlap with the antrum of a PV annulus section being treated. In case of a reflector the space between the piezoelectric or PZT transducer elements and the reflector communicates with an interior cooling fluid filled space 206 within balloon 109 which provides additional cooling for the transducer 112. Metallic coatings (see 72, 74, FIG. 6) on the interior and exterior surfaces of the array elements (or front and back in case of a planar design) serve as excitation or poling electrodes and are connected to a ground wire 208 and a signal wire 210 which extend through a wiring support tube to the distal end of the catheter. The wires 208 and 210 are connected to an ultrasonic excitation source 115 (FIG. 10) and a console or monitor 213 of an ultrasound imaging system. The process of forming such cylindrical arrays is well known and described in the prior art, see Eberle U.S. Pat. No. 6,049,958.

Electrical connection of the piezoelectric elements of array 112 with generator 115 and an imaging display or monitor 213 of a control system 156 (FIG. 10) is best achieved through flex circuit strip lines. In order to reduce the line count, multiplexer IC's can be deployed at the distal end of catheter 105, preferably close to ultrasound array 112. (See 12, FIGS. 5 and 6.) Of advantage are multiplexer circuits directly deposited at the distal end of the strip lines in a staggered fashion to keep the catheter diameter small.

The interior space 206 within balloon 109 is connected to a circulation device 116 (FIG. 10) for circulating a liquid, preferably an aqueous liquid, from a liquid source or supply 211 through the balloon to cool the ultrasound transducer 112 in order to avoid blood coagulation. Circulation device 116 includes at least one pump. As further discussed below, during operation, the circulation device 116 continually circulates the aqueous fluid through the balloon 109 and maintains the balloon under a desired pressure and temperature.

Catheter 105 is deployed via a sheath 100 (FIG. 8) generally in the form of an elongated tube having a proximal end, a distal end and a proximal-to-distal axis. Sheath 100 is advanced over a guide-wire through femoral access into the right atrium. After a septal puncture has been performed the catheter 105 is advanced through the sheath 100 into the left atrium LA (FIG. 8).

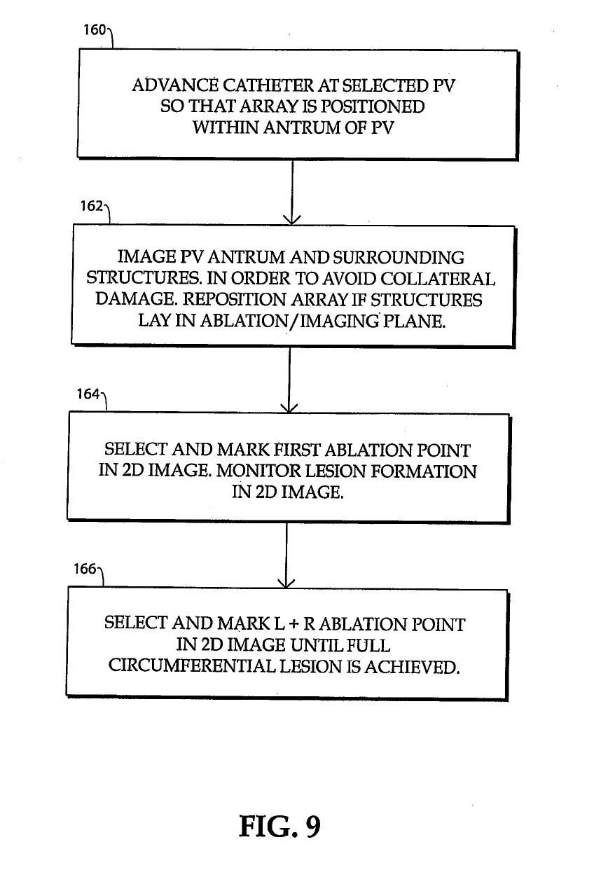

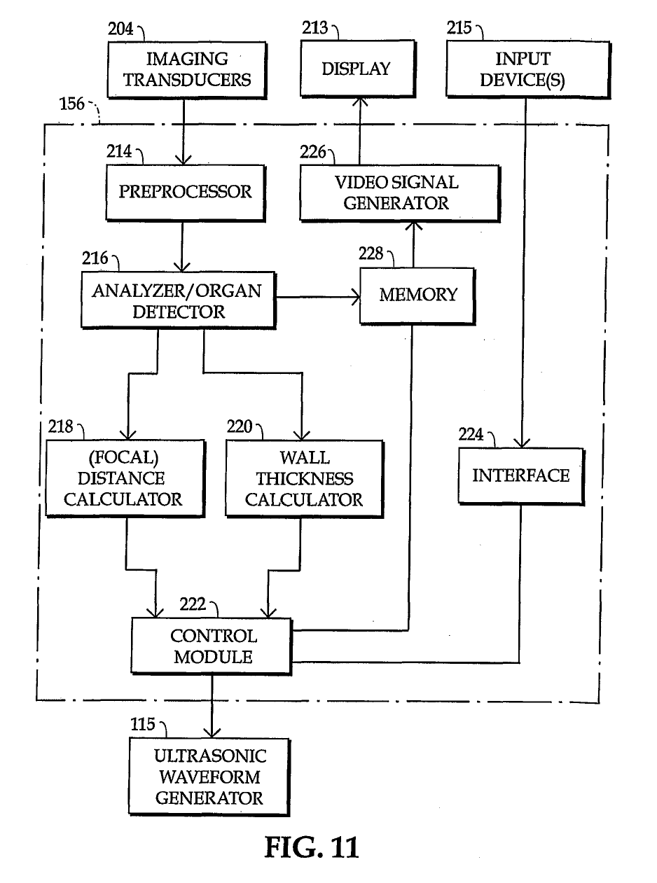

Treatment catheter 105 is advanced under ultrasound image guidance until the antrum of the selected pulmonary vein (PV) is clearly visualized. Treatment catheter is advanced further so that ultrasound transducer array 112 is positioned within the antrum of a selected pulmonary vein (PV) (step 160, FIG. 9). Ultrasound imaging guidance will reduce the need for fluoroscopic imaging and cut down on ionizing radiation. Once the treatment catheter has been positioned and mechanically stabilized by means of a sensing loop catheter 212 the ablation process can be controlled through the imaging system from the control room (steps 162, FIG. 9). Interactively ablation targets are identified in the image with markers (step 164, 166). The markers are instructions input to the control unit 156 (FIG. 11, or 56, FIG. 5), exemplarily via a touch screen (58, 213) or a keyboard and/or mouse input device (215), that indicate the location of a desired ablation on the organic structures represented in the displayed image. As discussed hereinafter in detail with reference to FIG. 11, the control system 156 translates these ablation markers into focusing, power and time parameters to control the ablation beam in the desired location and to ablate a lesion of the appropriate depth. During the ablation process the ablation site is monitored via ultrasound in an interlaced mode to allow the user to control the ablation process under essentially real time visualization. Since ablated tissue increases ultrasound reflectivity an intensity change can be observed during ablation. Ablated tissue clearly shows higher reflectivity than non ablated tissue so that the ablation can be terminated when a transmural lesion has been obtained.

With the catheter in the operative position, the energy field 114 (FIG. 7) is aligned with one point of the PV antrum image. In other words the therapy transducer section 202 is set under programming to focus ultrasonic vibration energy on the antrum wall at a particular location. The imaging transducer section 204 communicates, to the computer system control unit 156, ultrasonic waveform data from which the computer calculates distance of the therapy transducer section 202 from the atrial wall and the thickness of the atrial wall at the particular location of the antrum. More specifically, ultrasonic waveform generator 115 transmits an electrical signal of one or more pre-established ultrasonic frequencies to a selected transmitting transducer element of transducer array 112. Reflected ultrasonic waveform energy from internal organic structures of the patient is detected by sensor transducer elements of imaging transducer section 204 and processed by a preprocessor 214. Preprocessor 214 is connected to a signal analyzer 216 that computes dimensions and shapes of the internal organic structures. Output of analyzer 216 is organized and compared by a distance detector 218 to determine the distance of therapy transducer section 202 from the target location on the antrum or atrial wall, while an organ thickness detector 220 operates to compare echo signals to thereby determine the thickness of the pulmonary vein at the target location. Distance detector 218 and thickness detector 220 are connected to a therapy signal control module 222 that controls signal generator 115 to so energize the piezoelectric or PZT elements of therapy transducer section 202 in a phased array operation mode as to focus ultrasonic mechanical waves on the target location for a limited ablation time and power. Control module 222 may include a calculation submodule for determining the power and duration parameters of each ablation burst of ultrasonic mechanical waveform energy. The user can monitor the lesion formation in the ultrasound image on display console 213 and override the therapy system if so desired.

Control unit 156 includes an interface 224 for monitoring instructions input by the user via touch screen (60, 213) or keyboard and mouse (215). Signal analyzer 216 is connected to an image signal generator 226 that produces a video signal for display console 213 (or 60) and interface 224 is connected to control module 222 which interprets user directions in conjunction with the organic structures of the patient as detected, encoded and at least temporarily stored in memory 228 by analyzer 216.

As indicated above, ablation preferably in stepwise fashion around a circumferential locus defined by the user or surgeon via the input ablation markers. A neighboring ablation position is chosen as indicated in FIG. 9 and so on until a circumferential, continuous lesion has been created.

With the treatment catheter 105 and transducer array 112 in the operative position, the ultrasonic excitation source or waveform generator 115 actuates the therapy transducer section 202 of transducer array 112 to emit ultrasonic waves. Merely by way of example, the ultrasonic ablation waves (which are longitudinal compression waves) may have a frequency of about 1 MHz to a few tens of MHz, most typically about 8 MHz. The transducer typically is driven to emit, for example, about 10 watts to about 100 watts of acoustic power, most typically about 40 to 50 watts. The actuation is continued for about 10 seconds to about a minute or more, most typically about 20 seconds to about 40 seconds per lesion. Optionally, based on the ultrasound image the actuation may be repeated several times. The frequencies, power levels, and actuation times may be varied from those given above.

The various components of control unit 156 may be hard wired circuits designed to perform the specific computations discussed herein. Alternatively, control unit 156 may take the form of a generic microprocessor or computer with the components realized as generic digital circuits modified by programming to carry out the delineated functions.

The ultrasonic waves generated by the transducer array 112 propagate generally radially outwardly from the transducer elements, outwardly through the liquid within the balloon 109 to the wall of the balloon and then to the surrounding blood and tissue. The ultrasonic waves impinge on the tissues of the heart particularly on the PV antrum. Because all of the liquid within the balloon and the blood surrounding the balloon have approximately the same acoustic impedance, there is little or no reflection of ultrasonic waves at interfaces between the liquid within the balloon 109 and the blood outside the balloon.

Essentially all of the annulus within the PV antrum lies within the “near field” region of the transducer and particularly the therapy transducer section 202. Within this region, the outwardly spreading segmental beam 114 of ultrasonic waves tends to remain focused not only in the cross-sectional plane but also in elevation axis and has an axial length (the dimension of the beam along the catheter axis; see drawings in FIGS. 1 and 2) approximately equal to the axial length of the transducer section 202.

The ultrasonic energy applied by the therapy transducer section 202 is effective to heat and thus necrose a section of the annulus in the PV antrum. A circular lesion formed by a continuous series of sectional ablations creates a conduction block which may be confirmed through lack of PV potentials detected with the loop sensing catheter 212. (Catheter 212 carries a series of mutually spaced sensing electrodes 224 that detect voltage potentials in the cardiac tissue.) The circumferential lesion may take on a variety of shapes (oval or more complicated shapes) and depends on the surrounding anatomy of the PV antrum. The advantage of this approach is that all anatomical variations can be safely treated by moving the ablation plane axially to avoid ablating collateral structures and or by tilting the ablation plane by bending the distal portion of ablation catheter 105.

Numerous other variations and combinations of the features discussed above can be utilized without departing from the present invention as defined by the claims. For example, the emitter structure or transducer array 112 can be slideably mounted within the catheter so that the catheter stays in place during the treatment. In still other arrangements, several emitters might be mounted on the catheter in a chain like fashion in order to apply energy over the length of the catheter inserted into the left atrium. Again this configuration does not require a movement of the catheter during treatment. In still other embodiments, focusing devices, such as lenses and diffractive elements can be employed in case of ultrasonic energy.

The state of the lesion annulus within the PV antrum can be monitored by ultrasound imaging during the treatment. During treatment, the tissue changes its physical properties, and thus its ultrasound reflectivity when heated. These changes in tissue ultrasound reflectivity can be observed using ultrasonic imaging to monitor the formation of the desired lesion in the annulus within the PV antrum. Other imaging modalities which can detect heating can alternatively or additionally be used to monitor the treatment. For example, magnetic resonance imaging can detect changes in temperature. In the case of reliance on non-ultrasound imaging modalities, it is optional to include the imaging transducer section 204 as part of the ultrasound transducer array 112.

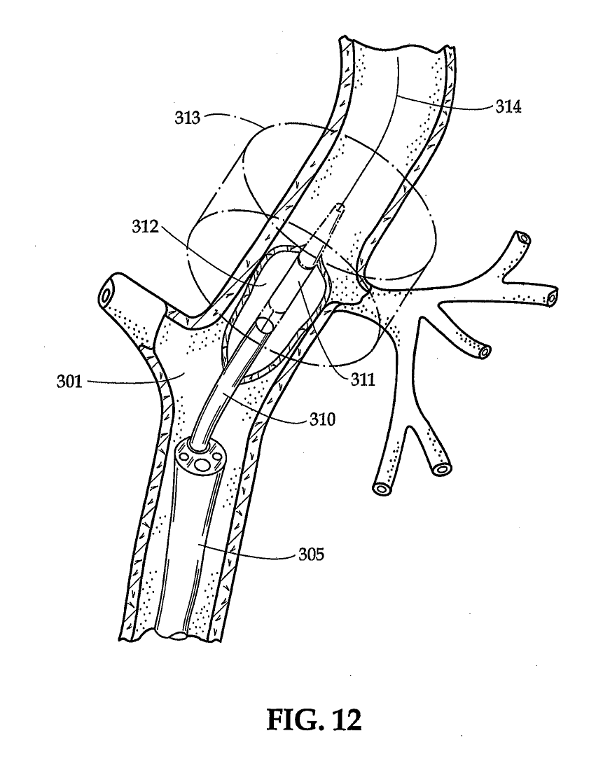

FIG. 12 depicts use, in the bronchial system, of a combined imaging and treatment catheter 310 as exemplarily described hereinabove with respect to catheter 5. Catheter 310 includes a composite or dual-mode transducer array 311 surrounded by a fluid-containing balloon 312. Catheter 311 is advanced through a bronchoscope 305 (or a sheath) and over a guide wire 314 into the right bronchial branch 301 and a portion of the transducer array 311 is activated to treat bronchial or lung tissues. The ultrasound treatment volume is indicated at 313. In its inflated condition, bladder 312 engages the bronchial wall and therewith allow for ultrasound to be conducted from transducer into the bronchial wall and surrounding tissues. Transducer array 311 is of a tubular shape and has an exterior composite emitting surface (an array of emitting surfaces) in the form of a cylindrical surface of revolution about the proximal-to-distal axis of the transducer array 311. The transducer array 311 typically has an axial length of approximately 2-10 mm, and preferably 6 mm. The outer diameter of the transducer array 311 is approximately 1.5-3 mm in diameter, and preferably 2 mm.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A method of treating atrial fibrillation (AF) of a mammalian subject, comprising the steps of:
(a) providing a dual mode therapy/imaging unit incorporating a plurality of electromechanical transducers adapted for producing and detecting ultrasonic vibrations and arranged in an at least partial cylindrical array;
(b) positioning said dual mode therapy/imaging unit within an antrum of a pulmonary vein (PV) to apply energy to one segment of a circular cross section proximal to the PV annulus; and
(b) repeatedly actuating said dual mode therapy/imaging unit to apply energy of about 50 to 100 W per square cm in a range of frequencies about 10 MHz to each of a plurality of portions of a circular cross section within the PV antrum until a complete circumferential lesion has been achieved.

2. A method as set forth in claim 1 wherein the positioning and actuating steps are performed while the heart is beating.

3. A method as set forth in claim 1, further comprising operating said dual mode therapy/imaging unit in an imaging mode to obtain ultrasound image data and operating a computer to display an ultrasound image from said data, wherein the step of actuating said dual mode therapy/imaging unit is performed for an ablation section selected from the displayed ultrasound image.

4. A method as set forth in claim 3, further comprising operating said computer to calculate therapeutic beam parameters including focal distance, ultrasound beam power and actuation duration and to actuate said dual mode therapy/imaging unit to necrose or ablate said portions of said circular cross section.

5. A medical apparatus comprising an elongate flexible tubular member provided along a distal end portion with an array of electromechanical transducers configured for dual mode ablation and imaging, said distal end portion including a sandwiched multilayer structure including said array as a first layer, and at least one impedance matching layer disposed over or atop said first layer.

6. The apparatus as set forth in claim 5, further comprising energizing circuitry operatively connected to said array for selectively activating said transducers as a phased array to focus ultrasound energy and obtain imaging data, said circuitry including multiplexer circuits disposed in a staggered fashion at or proximate said distal end portion.

7. An apparatus as set forth in 5 wherein said sandwiched multilayer structure includes, along part of an axial length thereof, reflective backing for therapeutic mode optimization and further includes, along another part of said axial length, absorptive backing for imaging mode optimization.

8. An apparatus as set forth in 5 wherein said array is in the form of a flat rotatable disc, divided into imaging and therapy portions respectively having absorptive and reflective backing.

9. Apparatus for isolating a pulmonary vein (PV) a mammalian subject comprising:
(a) a elongated catheter having proximal and distal regions;
(b) a emitter unit including an ultrasonic transducer and an expansible structure carried on the distal region of the catheter, the expansible structure being constructed and arranged to cool the transducer to avoid any blood coagulation.

10. Apparatus as set forth in claim 10 wherein the catheter includes a catheter steering mechanism carried on the catheter and operative to selectively bend a bend region of the catheter proximal to the emitter unit.

11. Apparatus as set forth in 10, further comprising a guide wire, the catheter being constructed and arranged so that the catheter can be advanced over the guide wire and the guide wire holding the ablation catheter in stable position so that the operator can control the PV isolation from the imaging console. The guide wire further serving as a loop sensing catheter to monitor electrically the isolation.

12. A minimally invasive surgical method comprising:
(a) providing a catheter assembly having a distal end portion carrying a balloon structure and an array of electromechanical transducer elements therein;
(b) inserting a segment of said catheter assembly into a patient so that said distal end portion is disposed inside a preselected tubular organ of the patient;
(c) inflating said balloon structure with a liquid;
(d) obtaining an image of internal organic structures of the patient in a region including said preselected tubular organ;
(e) positioning said distal end portion and said balloon structure in said preselected tubular organ; and
(f) activating said array to necrose or ablate a section of an inner surface of said preselected tubular organ to a controlled and limited depth so as to avoid necrosing tissues of adjacent organic structures.

13. A method as set forth in claim 12 wherein said section of said inner surface is an annular or circumferential area, and wherein the activating of said array includes controlling focal direction and range to necrose or ablate said section.

14. A method as set forth in claim 13 wherein the obtaining of said ultrasound image includes operating a computer to display said image in visually detectible format on a monitor or screen, further comprising operating an input device in conjunction with the display of said image to identify said section to said computer.

15. A method as set forth in claim 14, further comprising operating said computer to calculate therapeutic beam parameters including focal distance, ultrasound beam power and activation duration and to activate or energize said array to necrose or ablate said section.

16. A method as set forth in claim 12 wherein said image is an ultrasound image and said array is selectively configured for dual mode operation including imaging and therapeutic ablation, the obtaining of said image including poling transducer elements of said array to detect reflected ultrasonic pressure waves.

17. A method as set forth in claim 12 withdrawing the catheter assembly approximately one transducer length and again activating said array to necrose or ablate an additional section of said inner surface of said preselected tubular organ to a controlled and limited depth so as to avoid necrosing tissues of adjacent organic structures.

18. A method as set forth in claim 12 wherein said tubular organ is a pulmonary vein, said distal end portion being inserted into an antrum of the pulmonary vein, the method serving in the treatment of atrial fibrillation.

19. A method as set forth in claim 12 wherein the ultrasound transducer array is a therapeutic transducer array only and the obtaining of said image includes operating an MRI imaging device.

20. A method as set forth in claim 12 wherein said tubular organ is the lower esophageal sphincter, the method serving in a treatment of gastro-esophageal reflux disorder (GERD).

21. The method as set forth in claim 20 wherein the activating of said array includes emitting ultrasound energy in a density sufficient to shrink collagen, which is about a tenth of the energy density required to ablate tissue at or around 10 MHz.

22. A method as set forth in claim 12 wherein said tubular organ is the urethra, the method serving in a treatment of urinary incontinence.

23. The method as set forth in claim 22 wherein the activating of said array includes emitting ultrasound energy in a density sufficient to shrink collagen, which is about a tenth of the energy density required to ablate tissue at or around 10 MHz.

24. The method as set forth in claim 22 where the energy emitted is sufficient to ablate prostate tissue at about 50 to 100 W per square centimeter at or around 10 MHz.

25. A method as set forth in claim 12 wherein said tubular organ is taken from the group consisting of the mitral annulus, the tricuspid annulus, the aorta or a peripheral vein, the method serving in a treatment of valve disease.

26. The method as set forth in claim 25 wherein the activating of said array includes emitting ultrasound energy in a density sufficient to shrink collagen, about 5 to 10 W per square cm at or around 10 MHz.

27. The method as set forth in claim 12 wherein said tubular organ is in the bronchial system, the method serving in a treatment of lung tumors.

28. The method as set forth in claim 27 wherein the activating of said array includes emitting ultrasound energy in a density sufficient to ablate lung tumors, about 50 to 100 W per square cm at or around 10 MHz.

29. The apparatus used in claim 27 wherein said tubular organ is a bronchial branch and wherein the fluid filled balloon in inflated condition occludes the bronchial branch.

30. A therapeutic medical method comprising the steps of:
(a) inserting an introducer sheath into a patient;
(b) positioning a distal end of said introducer sheath inside an organ of the patient, said distal end of said sheath being provided with an array of electromechanical transducer elements;
(c) advancing a treatment catheter through the imaging sheath so that a distal end of said treatment catheter protrudes into said organ from the distal end of said sheath;
(d) operating said catheter to perform an operation on said organ; and
(e) during the operating of said catheter, energizing at least one said transducer elements with an ultrasonic electrical waveform and sampling a plurality of said transducer elements to detect incoming reflected ultrasonic waves, to obtain real time guidance for the operating of said catheter.

31. A medical apparatus comprising:
an elongate tubular member or sheath configured for minimally invasive medical procedures, said sheath having a distal end provided with an array of electromechanical transducer elements; and
electrical transmission circuitry operatively connected to said transducer elements for enabling an energizing of at least one said transducer elements with an ultrasonic electrical waveform and a sampling of a plurality of said transducer elements to detect incoming reflected ultrasonic waves, to obtain real time imaging data.

32. The apparatus of claim 31 wherein the array is configured in two dimensional directions to obtain 3D ultrasound images.

33. The apparatus of claim 32 wherein said transducer elements are disposed in a 2D circumferential ultrasound array.

34. The apparatus of claim 32 wherein said transducer elements are disposed in a longitudinal 2D ultrasound imaging array.

35. The apparatus of claim 31, further comprising an additional array of additional electromechanical transducer elements and additional electrical transmission circuitry operatively connected to said additional transducer elements for enabling energization of said additional electromechanical transducer elements for ultrasound therapy.

36. The apparatus of claim 31 wherein said array is integrated isometrically at said distal end of said sheath.

37. The apparatus of claim 31 wherein at least one outer layer of said sheath is adapted as matching layer.

38. The apparatus of claim 31 wherein said electrical transmission circuitry includes multiplexers, said outer sheath layer serving as a matching layer and a flex circuit electrically connecting said array with said multiplexers.

39. The apparatus of claim 31 wherein said sheath has a lumen which, when filled with blood acts as an array backing.

40. The apparatus of claim 31 wherein said sheath is provided with an inner sheath layer configured as an ultrasound diffraction layer.

41. The apparatus of claim 31 wherein said sheath includes a steering mechanism, operative to selectively bend a said distal end of said sheath containing said array and thereby selectively changing an imaging plane.

42. The apparatus of claim 31 wherein said sheath includes side holes for acoustic coupling fluid injection into non-blood filled treatment spaces.

43. The apparatus of claim 31 wherein said array is mounted in axial fashion to allow for sideways directed phased array imaging planes.
